(19) United States
(12) Patent Application Publication
(10) Pub. No.: US 2023/0047431 A1
(43) Pub. Date: Feb. 16, 2023

(54) Composition for treating or diagnosing osteoarthritis targeting ACVR2B

(71) Applicant: AJOU UNIVERSITY INDUSTRY-ACADEMIC COOPERATION FOUNDATION, Suwon-si (KR)

(72) Inventor: Siyoung YANG, Suwon-si (KR)

(73) Assignee: AJOU UNIVERSITY INDUSTRY-ACADEMIC COOPERATION FOUNDATION, Suwon-si (KR)

(21) Appl. No.: 17/880,269

(22) Filed: Aug. 3, 2022

(30) Foreign Application Priority Data
Aug. 3, 2021 (KR) 10-2021-0101682

(51) Int. Cl. G01N 33/68; G01N 33/50; A61P 19/02; A61K 31/7088

(52) U.S. Cl. CPC G01N 33/6893; G01N 33/5023; A61P 19/02; A61K 31/7088; G01N 2800/105; G01N 2333/71

(57) ABSTRACT

The present disclosure relates to a composition for treating or diagnosing osteoarthritis targeting activin A receptor type 2B (ACVR2B). More specifically, the present disclosure provides ACVR2B as a biomarker for diagnosing osteoarthritis because it was found that expression and activity of ACVR2B were increased at an early stage of osteoarthritis, expression of Mmp3, Mmp13, and Cox2 that induce destruction of cartilage was increased thereby, and expression of the genes was suppressed by inhibition of ACVR2B expression. In addition, since it was found that osteoarthritis was alleviated by suppressing the expression of ACVR2B, the present disclosure provides an ACVR2B expression or activity inhibitor as a therapeutic agent for osteoarthritis.

### BACKGROUND

### 1. Field of the Invention

The present disclosure relates to a composition for treating or diagnosing osteoarthritis targeting ACVR2B.

### 2. Description of the Related Art

Osteoarthritis (OA) is a degenerative joint disease mainly caused by inhibition of cartilage ECM synthesis and promotion of cartilage destruction. The progression of osteoarthritis is attributable to many aging-related etiological risk factors and pathophysiological processes. Mechanical stress including joint instability and damage as well as aging-related factors that make people susceptible to osteoarthritis are potential mechanisms that induce osteoarthritis. These factors cause degradation of extracellular matrix (ECM) by matrix metalloproteinase (Mmp) due to activated biochemical pathways in chondrocyte, a unique cell type that produces various catabolic and anabolic factors, as well as cessation of ECM synthesis through dedifferentiation and apoptosis of chondrocytes. In particular, once cartilage tissues constituting the joint are damaged, it is not regenerated properly in vivo. When the cartilage tissues of the joint are damaged, daily activities are restricted with severe pain accompanied. When it becomes chronic, fatal osteoarthritis is induced, deteriorating normal life or occupational activities.

On the other hand, in the case of rheumatoid arthritis (RA), unlike osteoarthritis caused by destruction of chondrocytes and cartilage tissues, autoimmune response is known to be an important factor causing the progression of a disease. Rheumatoid arthritis is a chronic autoimmune disease characterized by inflammation and proliferation of synovial cells, and unlike osteoarthritis, it accompanies osteoporosis and erosion in bones around the joint. Rheumatoid arthritis is developed by inflammation in the synovial membrane that spreads to the joint capsule, ligament, and tendon and then infiltrates into the bone. Therefore, the cause and progression of osteoarthritis and rheumatoid arthritis are completely different, so are the treatment methods thereof.

Nonsteroidal anti-inflammatory drugs (NSAIDs), penicillamine, steroid hormones, TNF inhibitors, interleukin inhibitors, JAK inhibitors, and anti-CD related inhibitors are currently known therapeutic agents for rheumatoid arthritis, which are suitable to block inflammatory mechanisms. Although NSAID drugs and steroid hormones are used for osteoarthritis patients to relieve joint pain and inflammation, they are hardly practical treatment for osteoarthritis since they relieve only symptoms rather than curing the disease itself. Furthermore, the cause and symptoms of osteoarthritis that is mainly caused by destruction of cartilage cells and cartilage tissues are fundamentally different from that of rheumatoid arthritis which is inflammatory arthritis. Thus, the treatment method for osteoarthritis should be inevitably different from that of rheumatoid arthritis.

Therefore, in order to prevent or delay the progression of osteoarthritis, early diagnosis is required. However, until now, only methods such as an orthopedic hospital visit or X-ray are known as methods for diagnosing osteoarthritis.

### PRIOR ART DOCUMENT

[Patent Document]

Korean Patent Application Publication No. 10-2016-0104905 (published on Sep. 6, 2016)

### SUMMARY

An object of the present disclosure is to provide a biomarker composition for diagnosing osteoarthritis early by checking changes in the expression or activity level of ACVR2B, a diagnostic kit capable of measuring the expression level of the biomarker, a method of diagnosing osteoarthritis using the biomarker, and a method of screening a therapeutic agent for osteoarthritis.

In addition, an object of the present disclosure is to provide a pharmaceutical composition for preventing or treating osteoarthritis, containing an ACVR2B expression or activity inhibitor as an active ingredient.

Example embodiments of the present disclosure provide a biomarker composition for diagnosing osteoarthritis, including an ACVR2B gene or a protein encoded by the gene.

Example embodiments of the present disclosure provide a kit for diagnosing osteoarthritis, containing, as an active ingredient, an agent for detecting an expression or activity level of an ACVR2B gene or a protein encoded by the gene.

Example embodiments of the present disclosure provide a method of providing information for diagnosis of osteoarthritis, including checking an expression or activity level of an ACVR2B gene or a protein encoded by the gene from a sample isolated from a specimen.

Example embodiments of the present disclosure provide a method of screening a therapeutic agent for osteoarthritis, including treating chondrocytes isolated from an osteoarthritis patient with candidate material; checking an expression level of activin A receptor type 2B (ACVR2B) from the chondrocytes treated with the candidate material; and providing the candidate material as a therapeutic agent for osteoarthritis when the expression level of ACVR2B of the candidate material is reduced compared to a group untreated with the candidate material.

Example embodiments of the present disclosure provide a pharmaceutical composition for preventing or treating osteoarthritis, containing an ACVR2B expression or activity inhibitor as an active ingredient.

In addition, example embodiments of the present disclosure provide a health food for preventing or alleviating osteoarthritis, containing an ACVR2B expression or activity inhibitor as an active ingredient.

According to example embodiments of the present disclosure, it was found that expression and activity of ACVR2B were increased at an early stage of osteoarthritis, expression of Mmp3, Mmp13, and Cox2 that induce destruction of cartilage was increased thereby, and expression of the genes was suppressed by inhibition of ACVR2B expression. Thus, example embodiments of the present disclosure provide ACVR2B as a biomarker for diagnosing osteoarthritis. Since it was found that osteoarthritis is alleviated by suppressing expression of ACVR2B, example embodiments of the present disclosure provide the ACVR2B expression or activity inhibitor as a therapeutic agent for osteoarthritis.

### BRIEF DESCRIPTION OF THE DRAWINGS

FIG. 1 shows results identifying that ACVR2B is expressed along with Mmp3, Mmp13, and Cox2 that induce joint destruction in joint tissues of osteoarthritis patients and osteoarthritis-induced mice. FIG. 1A shows results identified in a human joint, and FIG. 1B in a mouse joint.

FIG. 2 shows results identifying that the expression of ACVR2B is increased from the moment osteoarthritis is induced, and the expression of Mmp3, Mmp13, and Cox2 is gradually increased while cartilage destruction is induced.

FIGS. 3A and 3B show results identifying that the expression of ACVR2B is suppressed by injecting adenovirus ACVR2B shRNA into the knee joint induced with osteoarthritis via DMM in an attempt to knockdown ACVR2B so that osteoarthritis is alleviated.

FIG. 4A shows results of immunohistochemistry analysis identifying that ACVR2B is increased as osteoarthritis gradually progresses, FIG. 4B shows results identifying that osteoarthritis is suppressed when soluble FC-ACVR2B is injected into knee joint induced by osteoarthritis.

### DETAILED DESCRIPTION

Hereinafter, an example embodiment of the present disclosure will be described in more detail.

Since it was found that expression and activity of ACVR2B was increased at an early stage of osteoarthritis, expression of Mmp3, Mmp13, and Cox2 that induce destruction of cartilage was increased thereby, and expression of the genes was suppressed by inhibition of ACVR2B expression, the inventors of an example embodiment of the present disclosure completed the present disclosure by providing ACVR2B as a biomarker for osteoarthritis to diagnose osteoarthritis early and enhance therapeutic effects of osteoarthritis by regulating expression or activity of ACVR2B.

An example embodiment of the present disclosure may provide a biomarker composition for diagnosing osteoarthritis, including an ACVR2B gene or a protein encoded by the gene.

The activin A receptor type 2B (ACVR2B) may have an NCBI accession number. NP_001097.2.

An example embodiment of the present disclosure may provide a kit for diagnosing osteoarthritis, containing, as an active ingredient, an agent for detecting an expression or activity level of an ACVR2B gene or a protein encoded by the gene.

The agent may be selected from the group consisting of a primer or probe that specifically binds to an ACVR2B gene and an antibody, peptide, aptamer, or compound that specifically binds to an ACVR2B protein.

An example embodiment of the present disclosure may provide a method of providing information for diagnosis of osteoarthritis, including checking an expression or activity level of an ACVR2B gene or a protein encoded by the gene from a sample isolated from a specimen.

The sample may be selected from the group consisting of cells, tissues, blood, serum, urine, and saliva.

The method of providing information for diagnosis of osteoarthritis may be to diagnose as osteoarthritis when the expression level of an ACVR2B gene or a protein encoded by the gene is increased compared to a normal control group.

An example embodiment of the present disclosure may provide a method of screening a therapeutic agent for osteoarthritis, including treating chondrocytes isolated from an osteoarthritis patient with candidate material; checking an expression level of activin A receptor type 2B (ACVR2B) from the chondrocytes treated with the candidate material; and providing the candidate material as a therapeutic agent for osteoarthritis when the expression level of ACVR2B of the candidate material is reduced compared to a group untreated with the candidate material.

Methods for checking the expression level may be one or more methods selected from the group consisting of Western blotting, enzyme linked immunosorbent assay (ELISA), radioimmunoassay (RIA), radial immunodiffusion, Ouchterlony immunodiffusion, rocket immunoelectrophoresis, immunohistochemical staining, immunoprecipitation assay, complement fixation assay, immunofluorescence, immunochromatography, fluorescence activated cell sorter (FACS) analysis, and protein chip technology, but are not limited thereto.

The term “biomarker” as used herein refers to a substance capable of diagnosing tissues or cells of a subject suspected of osteoarthritis by distinguishing the tissues or cells from those of a normal control group, and includes organic biomolecules such as proteins or nucleic acids, lipids, glycolipids, or glycoproteins showing an increase or decrease in tissues or cells of the subject compared to the normal control group.

The term “diagnosis” as used herein refers to, in a broad sense, determining of the actual condition of a disease of a patient in all aspects. The content of determination may include disease name, cause of a disease, disease type, severity, detailed condition of a disease, and the presence or absence of complications.

An example embodiment of the present disclosure may provide a pharmaceutical composition for preventing or treating osteoarthritis, containing an ACVR2B expression or activity inhibitor as an active ingredient.

The ACVR2B expression inhibitor may be one or more selected from the group consisting of an antisense nucleotide, small interfering RNA (siRNA), short hairpin RNA (shRNA), microRNA, and ribozyme that complementarily bind to an ACVR2B gene or an mRNA of a gene that promotes expression of ACVR2B.

The ACVR2B activity inhibitor may be one or more selected from the group consisting of a protein that promotes ACVR2B activity or a compound, peptide, peptidomimetic, protein, aptamer, and antibody that that specifically bind to the ACVR2B protein.

In an example embodiment of the present disclosure, any one formulation selected from the group consisting of injections, granules, powder, tablets, pills, capsules, suppositories, gels, suspensions, emulsions, drops, or liquids may be used in the pharmaceutical composition according to a conventional method.

In another example embodiment of the present disclosure, the pharmaceutical composition for preventing or treating osteoarthritis containing an ACVR2B expression or activity inhibitor as an active ingredient may further include one or more additives selected from the group consisting of carriers, excipients, disintegrants, sweeteners, coating agents, swelling agents, lubricants, flavoring agents, antioxidant, buffers, bacteriostats, diluents, dispersants, surfactants, binders, and lubricants that are appropriate to be commonly used for the manufacture of pharmaceutical compositions.

Specifically, used as carriers, excipients, and diluents may be lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil. Tablets, pills, powder, granules, and capsules are included in the solid preparations for oral administration, and these solid preparations may be prepared by mixing at least one excipient such as starch, calcium carbonate, sucrose or lactose, and gelatin in the composition. In addition to simple excipients, lubricants such as magnesium stearate and talc may also be used. Liquid formulations for oral administration may include suspensions, solutions, emulsions, and syrups. Also, various excipients such as wetting agents, sweeteners, fragrances, and preservatives may be included in addition to water and liquid paraffin which are commonly used simple diluents. Formulations for parenteral administration include sterile aqueous solutions, non-aqueous solutions, suspensions, emulsions, freeze-dried preparations, and suppositories. Propylene glycol, polyethylene glycol, vegetable oil such as olive oil, and injectable esters such as ethyl oleate may be used as non-aqueous solvents and suspensions. As a base material for the suppository, witepsol, macrogol, tween 61, cacao butter, laurin, and glycerogelatin may be used.

According to an example embodiment of the present disclosure, the pharmaceutical composition may be administered to a subject in a conventional manner via intravenous, intraarterial, intraperitoneal, intramuscular, intrasternal, transdermal, intranasal, inhalational, topical, rectal, oral, intraocular, or intradermal routes.

The preferred dose of the ACVR2B expression or activity inhibitor may vary depending on the condition and weight of a subject, the type and extent of a disease, the drug form, the route and duration of administration, and may be appropriately selected by those skilled in the art. According to an example embodiment of the present disclosure, although not limited thereto, the daily dose may be 0.01 to 200 mg/kg, specifically 0.1 to 200 mg/kg, and more specifically 0.1 to 100 mg/kg. Administration may be conducted once a day or in several divided doses, but the scope of an example embodiment of the present disclosure is not limited thereby.

In an example embodiment of the present disclosure, the term “subject” as used herein may refer to a mammal including human, but is not limited to the examples.

In addition, an example embodiment of the present disclosure may provide a health food for preventing or alleviating osteoarthritis, containing an ACVR2B expression or activity inhibitor as an active ingredient.

The health food is used together with other foods or food additives other than the ACVR2B expression or activity inhibitor, and may be appropriately applied according to a conventional method. The mixed amount of the active ingredient may be appropriately determined according to the purpose of use thereof, for example, prophylactic, health, or therapeutic treatment.

The effective dose of the compound contained in the health food may be applied according to the effective dose of the therapeutic agent, but in the case of long-term intake for health and hygiene or health control, the dose may be less than or equal to the above range. It is clear that the active ingredient may be used in an amount beyond the above range since there is no problem in terms of safety.

The type of health food is not particularly limited, and examples include meat, sausage, bread, chocolate, candy, snacks, confectionery, pizza, ramen, other noodles, gum, dairy products including ice cream, various soups, beverages, tea, drinks, alcoholic beverages, and vitamin complexes.

Hereinafter, to help the understanding of the present disclosure, example embodiments will be described in detail. However, the following example embodiments are merely illustrative of the content of an example embodiment of the present disclosure, and the scope of the present disclosure is not limited to the following example embodiments. Example embodiments of the present disclosure are provided to more completely explain the present disclosure to those of ordinary skill in the art.

### Experimental Example

The following experimental examples are intended to provide experimental examples commonly applied to each example embodiment according to an example embodiment of the present disclosure.

1. Preparation of Human Osteoarthritis Samples and Experimental Osteoarthritis Mouse Models

Human cartilage samples were obtained from individuals with the age of 63 to 80 who underwent total knee arthroplasty (Table 1). All patients provided written consent, and sample collection was approved by the IRB of the Catholic University of Korea (UC14CNSI0150).

Male C57BL/6 was maintained according to the guidelines of the Institutional Animal Care and Use Committee, which approved all animal procedures at Laboratory Animal Research Center in Ajou University.

TABLE 1

| | Age/ | ICRS^{a} | | Weight | Height | BMI^{b} | |
|---|---|---|---|---|---|---|---|
| No. | gender | grade | Joint | (kg) | (m) | (kg/m²) | Use |
| 1 | 65/F | 4 | Knee | 158 | 53 | 21.23 | IHC/qPCR |
| 2 | 80/F | 4 | Knee | 143.3 | 55.1 | 26.83 | IHC/qPCR |
| 3 | 72/F | 4 | Knee | 165 | 65 | 23.88 | IHC/qPCR |
| 4 | 63/F | 4 | Knee | 152 | 52 | 22.51 | IHC/qPCR |
| 5 | 69/F | 4 | Knee | 151 | 60 | 26.31 | IHC/qPCR |
| 6 | 73/F | 4 | Knee | 153.8 | 70.75 | 29.89 | IHC |
| 7 | 63/F | 4 | Knee | 156 | 72.2 | 29.67 | IHC |
| 8 | 73/F | 4 | Knee | 154 | 83 | 35 | IHC |
| 9 | 75/F | 4 | Knee | 154 | 55 | 23.19 | IHC |
| 10 | 63/F | 4 | Knee | 163 | 74.3 | 27.96 | IHC |

^{a}ICRS, International Cartilage Repair Society;
^{b}BMI, Body Mass Index

To prepare an osteoarthritis models, 12-week-old male mice were subjected to surgical destabilization of the medial meniscus (DMM) and sacrificed 10 weeks post-surgery. Female mice were excluded from the experiment due to the influence by female hormones on the cause of osteoarthritis. Adenoviruses for intra-articular injection were purchased from Vector Biolabs (Malvern, USA): Ad-C (1060), Ad-shACVR2B (shADV-252021).

Adenovirus (1×10⁹ PFUs/10 μL) was injected into the knee joint of wild-type mice two times every week, and the mice were sacrificed three weeks after the first adenovirus injection. FC-sACVR2B (human FC-sACVR2B: Gene ID 93) injected intra-articularly was purchased from Y Biologies (Korea): 10 μg of FC-sACVR2B (Cat #AR-0114) was injected once every week for six weeks from the fourth week after the DMM.

2. Reagents

Antibodies were purchased from Enzo Biochem (New York, USA; ACVR2B) and Abcam (COX2, MMP3, MMP13).

3. Histology and Immunohistochemistry

Human osteoarthritis cartilage and mouse knee joint were fixed in 4% paraformaldehyde and embedded in paraffin. Mouse knee joints were calcified in 0.5 M EDTA (pH 7.4) for 2 weeks. Paraffin-embedded samples were stained with safranin-O, Alcian blue, or immunostaining. Cartilage destruction was assessed in experimental osteoarthritis mouse models by three observers who were unaware of the experimental grouping and scored according to the OARSI (Osteoarthritis Research Society International) grading system (grade 0-6). OARSI scores were presented as the mean maximal score for each mouse. Representative safranin-O staining images were selected from the most advanced lesions in each section, and osteophyte maturity was quantified as previously described. Subchondral bone sclerosis was determined by measuring the thickness of subchondral osseous lamina. Immunohistochemical staining was performed on human and mouse cartilage sections using MMP3, MMP13, COX2, and ACVR2B antibodies.

4. Statistical Analysis

All experiments were performed independently four or more times. Two independent groups were subjected to comparison using Shapiro-Wilk normality test, Levene's homogeneity of variance test, and two-tailed independent t-test. Multiple comparisons were performed using Shapiro-Wilk test, Levene's test, and one-way analysis of variance with Bonferroni's post-hoc test. Data based on the ordinal grading system was analyzed using non-parametric Mann-Whitney U tests. P values less than 0.05 were considered statistically significant.

### <Example 1> Identification of Correlation Between Changes in the Gene Expression Pattern Due to ACVR2B Overexpression and Osteoarthritis

To determine a possible role of ACVR2B in the joint, ACVR2B expression was investigated in human and mouse cartilage samples damaged or undamaged by osteoarthritis.

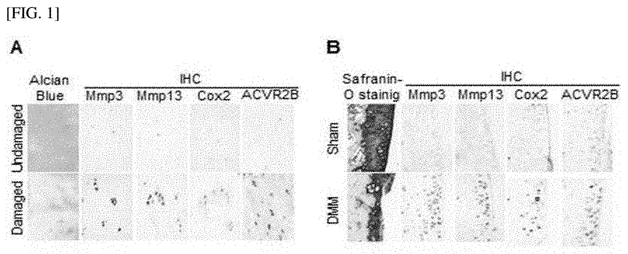

As a result, it was found that expression of Mmp3, Mmp13, and Cox2 was increased in the damaged osteoarthritis cartilage as shown in FIG. 1 compared to the undamaged sample, and at the same time, it was also found that the expression of ACVR2B was remarkably high (FIG. 1A; human, FIG. 1B; mouse).

Next, in order to find the expression time point of ACVR2B, after osteoarthritis was induced in mice by DMM, expression patterns of ACVR2B, Mmp3, Mmp13, and Cox2 and cartilage destruction were identified by immunohistochemistry and safranin-O staining. At the same time, the time point of onset of osteoarthritis was identified by checking the degree of cartilage destruction via OARSI grade, osteophyte maturity, and SBP thickness.

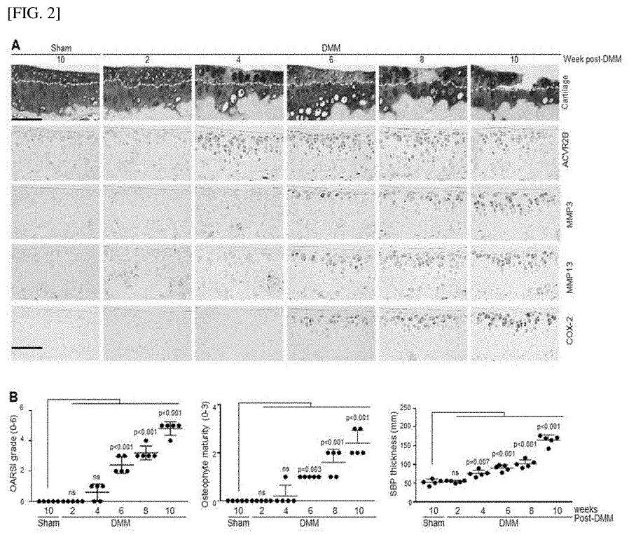

As shown in FIGS. 2A and 2B, as a result of identification through safranin-O staining, immunohistochemistry, and various scoring, it was found that osteoarthritis occurred from the sixth week after induction by DMM, and at the same time, expression of Mmp3, Mmp13, and Cox2 was increased from the sixth week. However, in the case of ACVR2B, it was found that expression was observed through immunohistochemistry before the expression of Mmp3, Mmp13, and Cox2 began.

From the above results, it was found that the expression of Mmp3, Mmp13, and Cox2, that induce cartilage degeneration, may be regulated by ACVR2B.

### <Example 2> Identification of Suppressive Effect of Osteoarthritis Through Suppression of ACVR2B Expression

Since the expression of ACVR2B increases from the fourth week in mice induced with osteoarthritis by DMM, Ad-shACVR2B was injected to the mouse joints induced with osteoarthritis by DMM via intra-articular injection after DMM surgery, once every week from the fourth week.

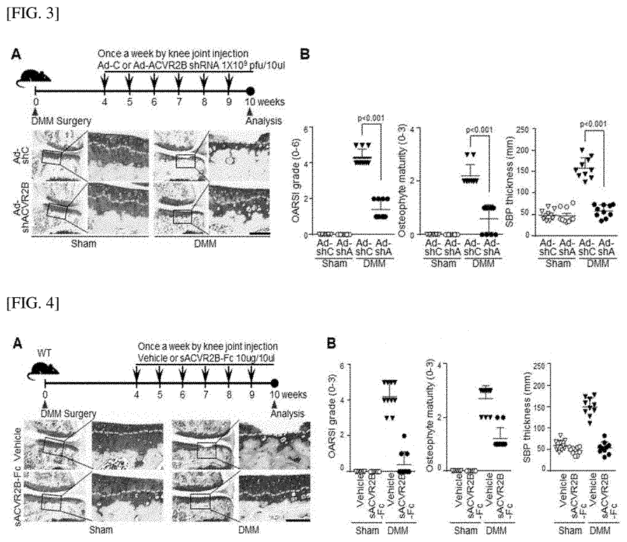

As a result, as shown in FIGS. 3A and 3B, it was found that osteoarthritis was attenuated as ACVR2B was not expressed.

From the above results, it may be suggested that ACVR2B plays an important role in the development of osteoarthritis.

### <Example 3> Identification of Correlation of Osteoarthritis Development by Inhibition of ACVR2B Activity

As observed in the previous experiment, the expression of ACVR2B was increased from the fourth week in mice induced with osteoarthritis by DMM, and the expression of Mmp3, Mmp13, and Cox2 was increased from the sixth week. Thus, it was found that expression of Mmp3, Mmp13, and Cox2 may be regulated by ACVR2B activity.

In addition, the soluble activin receptor FC-ACVR2B was administered once every week from the fourth week after DMM surgery in order to trap various ligands capable of binding to ACVR2B under osteoarthritis-induced conditions.

As a result, it was found that the induction of osteoarthritis was suppressed as shown in FIGS. 4A and 4B.

From the above results, it was found that regulation of ACVR2B sub-targeting factors was suppressed because the binding of ACVR2B ligand to ACVR2B was fundamentally blocked by soluble FC-ACVR2B, leading to inhibition of ACVR2B activity.

As specific parts of the present disclosure have been described above in detail, it is apparent for those skilled in the art that these specific descriptions are only preferred example embodiments and that the scope of the present disclosure is not limited thereby. Accordingly, the substantial scope of an example embodiment of the present disclosure will be defined by the appended claims and equivalents thereof.

1-3. (canceled)

4. A method of providing information for diagnosis of osteoarthritis, the method comprising checking an expression or activity level of an activin receptor type 2B (ACVR2B) gene or a protein encoded by the gene from a sample isolated from a specimen.

5. The method of claim 4, wherein the sample is selected from the group consisting of cells, tissues, blood, serum, urine, and saliva.

6. The method of claim 4, wherein the method comprises diagnosing as osteoarthritis when the expression or activity level of the ACVR2B gene or a protein encoded by the gene increases compared to a normal control group.

7. A method of screening a therapeutic agent for osteoarthritis, comprising:
treating chondrocytes isolated from an osteoarthritis patient with candidate material;
checking an expression level of activin receptor type 2B (ACVR2B) from the chondrocytes treated with the candidate material; and
providing the candidate material as a therapeutic agent for osteoarthritis when the expression level of ACVR2B of the candidate material is reduced compared to a group untreated with the candidate material.

8. A pharmaceutical composition for preventing or treating osteoarthritis, comprising an activin receptor type 2B (ACVR2B) expression or activity inhibitor as an active ingredient.

9. The pharmaceutical composition of claim 8, wherein the ACVR2B expression or activity inhibitor is one or more selected from the group consisting of an antisense nucleotide, siRNA, shRNA, microRNA, and ribozyme that specifically bind to an ACVR2B gene or an mRNA of a gene that promotes expression of ACVR2B.

10. The pharmaceutical composition of claim 8, wherein the ACVR2B expression or activity inhibitor is one or more selected from the group consisting of a compound, peptide, peptidomimetic, protein, aptamer, and antibody that specifically bind to a protein that promotes activity of ACVR2B or an ACVR2B protein.

11. (canceled)
